(19) United States
(12) Patent Application Publication
(10) Pub. No.: US 2003/0170326 A1
(43) Pub. Date: Sep. 11, 2003

(54) Bioavailability enchancing activity of Zingiber officinale Linn and its extracts/fractions thereof

(75) Inventors: Ghulam Nabi Qazi, Jammu (IN); Kasturi Lal Bedi, Jammu (IN); Rakesh Kamal Johri, Jammu (IN); Manoj Kumar Tikoo, Jammu (IN); Ashok Kumar Tikoo, Jammu (IN); Subhash Chander Sharma, Jammu (IN); Sheikh Tasaduq Abdullah, Jammu (IN); Om Parkash Suri, Jammu (IN); Bishan Datt Gupta, Jammu (IN); Krishan Avtar Suri, Jammu (IN); Naresh Kumar Satti, Jammu (IN); Ravi Kant Khajuria, Jammu (IN); Surjeet Singh, Jammu (IN); Anamika Khajuria, Jammu (IN); Bal Krishan Kapahi, Jammu (IN)

Correspondence Address: LADAS & PARRY, 26 WEST 61ST STREET, NEW YORK, NY, 10023, US

(73) Assignee: COUNCIL OF SCIENTIFIC AND INDUSTRIAL RESEARCH

(21) Appl. No.: 10/318,314

(22) Filed: Dec. 12, 2002

Related U.S. Application Data
(60) Provisional application No. 60/341,174, filed on Dec. 13, 2001.

(51) Int. Cl. A61K 35/78; A61K 31/7048; A61K 31/704; A61K 31/496; A61K 31/4709; A61K 31/545; A61K 31/43

(52) U.S. Cl. 424/734; 424/756; 514/36; 514/29; 514/200; 514/192; 514/253.08; 514/312

(57) ABSTRACT

The present invention relates to a bio enhancing composition containing extract and/or bioactive fraction/isolate from the plant Zingiber officinale in combinations combination with drugs, nutrients, nutraceuticals, micronutrients and herbal drugs/products and optionally containing piperine as a extract/active fraction obtained from piper nigrum, piper longum or its oleoresin as a bioavailability enhancer and its process for producing the extract or fractions from the plant source.

### FILED OF THE INVENTION

The present invention relates to a composition containing extract and/or bioactive fraction/isolate from the plant Zingiber officinale as a bioavailability enhancer. The present invention also relates to a composition containing extract and/or bioactive fraction/isolate from the plant Zingiber officinale with piperine as a bioavailability enhancer.

### BACKGROUND AND PRIOR ART REFERENCES

Ginger of commerce or ‘Adrak’ is the dried underground stem or rhizome of the zingiberous, herbaceous plant Zingiber officinale Linn, which constitutes one of the five most important major spices of India, standing 3rd or 4th, competing with chillies, depending upon fluctuations in world market prices and world demand and supply positions. Ginger ranks third in value among all the spices exported from India, being next to pepper and cardamom. Indian ginger is further classified as (i) Malabar ginger (Kerala), (ii) Cochin ginger or Wyanad ginger (iii) Himachal ginger, and (iv) Sikkim/N.E. region ginger.

Ginger is valued in medicine as a carminative and stimulant to the gastro-intestinal tract. It is much in vogue as a household remedy for flatulence and colic. Externally, ginger is used as a local stimulant and rubefacient. It is included among anti-depressants and it forms an ingredient of some anti-narcotic preparations. Besides its stimulant and carminative properties, it is used in toothaches, gout and rheumatism. The essence of ginger is used to promote digestion. Ginger is reported to act powerfully on the mucous membrane. Beneficial results have been obtained when it has been administered in pulmonary and catarrhal affections. Externally, ginger has been used as curative for headaches, paralysis and rheumatism and internally with other ingredients in intermittent fevers (Wealth of India, Raw Material vol XI (1976), 89, PID, CSIR, New Delhi).

Based on our past experience with the development of piperine as bioavailability enhancer from plants which are part of human diet as well as being medicinal, we took up this plant to evaluate its bioavailability enhancing effect, if any, based on its attributes bearing some similarities to the plant sources of piperine.

Several approaches have been adopted in the past to maximize oral bioavailability, such as (a) particle size reduction (micronization, nanonization, etc.,) (b) polymorphic or crystal size and form selection, (c) solubilization of lesser soluble drugs by way of chemical modifications, complexation and use of co-solvents/surfactants, (d) targeted delivery of drug at the site of action, (e) controlled drug delivery by film coating or use of polymeric matrices for sustained release of drugs, (f) prodrug approach, and (g) microencapsulation using liposomes.

However, based on information from Ayurvedic literatures, a new approach of increasing the bioavailability of drugs including poorly bioavailable drugs had been conceptualized at RRL, Jammu. One of the groups of herbals which has been documented very frequently as essential part of about 70% of Ayurvedic prescriptions, was noted to be ‘Trikatu’, that comprises three acrids viz. long pepper, black pepper and dry ginger in equal proportions. A single major alkaloidal constituent from peppers (piperine) was found to be responsible for bioavailability enhancing effect. The role of ginger is to regulate intestinal function to facilitate absorption. Influence of piperine was extensively studied on anti-TB drugs. It was determined that in combination with piperine the dose of rifampicin can be reduced by about 50% while retaining the therapeutic efficacy of this anti-TB drug at par with the standard dose (450 mg). Based on these findings several other reputed plants were evaluated for bioavailability/bioefficacy enhancing activity. Polar and non-polar extracts of parts of a few plants viz., Cuminum cyminum and Carum carvi increased significantly (25-435%), the bioavailability of a number of classes of drugs, for example, but not limited to, antibiotics, antifungals, anti-virals, anticancer, cardiovascular, CNS, anti-inflammatory/anti-arthritic, anti-TB/antileprosy, anti-histaminic/respiratory disorders, corticosteroids, immunosuppressants, anti-ulcer. Such extracts either in presence or absence of piperine have been found to be highly selective in their bioavailability/bioefficacy enhancing action.

U.S. Pat. No. 5,972,382 by Majeed, et al titled as “Use of piperine as a bioavailability enhancer” discloses compositions and methods for the improvement of gastrointestinal absorption and systemic utilization of nutrients and nutritional supplements, wherein the compositions comprise a minimum of 98% of pure alkaloid piperine. The method comprises oral, topical, or parenteral administration of the compositions of the invention. A new process for the extraction and purification of piperine is also disclosed.

U.S. Pat. No. 5,536,506 by Majeed, et al. titled as “Use of piperine to increase the bioavailability of nutritional compounds” discloses a new composition and method for the improvement of gastrointestinal absorption and systemic utilization of nutrients and nutritional supplements, wherein the composition comprises an extract from the fruit of Piper containing a minimum of 98% of pure alkaloid piperine. The method comprises oral, topical, or parenteral administration of the compositions of the invention. A new process for the extraction and purification of piperine is also disclosed.

Another U.S. Pat. No. 5,744,161 by Majeed, et al. titled as “Use of piperine as a bioavailability enhancer” discloses compositions and methods for the improvement of gastrointestinal absorption and systemic utilization of nutrients and nutritional supplements, wherein the compositions comprise a minimum of 98% of pure alkaloid piperine. The method comprises oral, topical, or parenteral administration of the compositions of the invention. A new process for the extraction and purification of piperine is also disclosed.

Chemistry of Zingiber officinale

On steam distillation, dried, cracked and comminuted ginger yields 1-3% (average 2%) of pale yellow, viscid oil. The oil possesses the aromatic odor but not the pungent flavor (bite) of the spice. Of course, the odor of the oil is quite lasting. The physico-chemical characteristics of the oil vary depending upon the variety, period of storage and method of extraction etc (E. Guenther, The Essential Oils vol v V. D. Van Nostrand Co., Inc, NY (1952),116). It is more economical and convenient to recover oil or oleoresin from dried ginger than from fresh ones. However, fresh scrapings of ginger are quite rich in oil and should be immediately used for steam distillation, otherwise 60-80% of volatile oil is lost. Ginger oleoresin is obtained by extraction of powdered dried ginger with suitable solvents like alcohol, acetone or any other efficient solvent. Unlike volatile oil, it contains both the volatile oil and non-volatile pungent principles for which ginger is so highly valued. Concentration of the solvent extracts under vacuum and on complete removal of even trace of solvent used yields the oleoresin of ginger. The quantitative composition of the oleoresin depends upon the solvent used. Ginger oleoresin (Gingerin) generally contains following types of compounds: Gingerols, Zingerones, Shogaols, volatile oil, resins, phenols etc. Ginger oleoresin is manufactured in India and abroad and is in great demand by the various food industries.

Chemical composition of ginger oil and oleoresin

The oil contains sesquiterpene hydrocarbons (50% or more), sesquiterpene alcohols, monoterpenoids and associated compounds, esters of acetic acid and caprylic acid and a trace of chavicol. The sesquiterpene hydrocarbons present are Zingiberene (α and β, ˜35.6%), ar-curcumene (17.7%), farnesene (9.8%) and relatively smaller amounts of β-bisabolene, γ-selinene, β-elemene and β-sesquiphellandrene. Oxygenated sesquiterpene constituents include zingiberol and two other isomeric alcohols. Monoterpene hydrocarbons present in the oil include camphene, α and β-pinene, cumene, myrcene, limonene, p-cymene and β-phellandrene. The oxygenated monoterpenes and associated compounds present are 2-heptanol, 2-nonanal, n-nonanal, n-decanal, methyl heptenone and 1,8-cineole. (M. C. Nigam, I. C. Nigam, L. Levi & K. L. Handa Can J. Chem 42 (1964), 2610)

Studies on the oils derived from ginger from Jamaica, Nigeria, Sierraleone, China, India and Australia have shown that the composition of the oils have similar patterns, although quantitative differences do exist. The content of zingiberenes lies in the range of 20-30%; the ranges of values for a few other constituents are: β-bisabolene, 5-12%; ar-curcumene, 6-19% and β-sesquiphellandrene, 7-12%. The citral (geranial and neral) content is highly variable; the Australian oils are rich in citral (8-27% av. 19.8%; a sample prepared from fresh rhizomes by extraction at r.t. contained 8.8% geranial and 1.5% neral).

The pungent principles of ginger are non-volatile. These can be extracted from coarsely ground-dried spice by using some suitable solvent. They mainly consist of oxymethyl phenols, the major components being gingerol, shogaol, zingerone and paradol. (D. W. Connell and M. D. Sutherland, Aust. J. Chem. Soc. 22 (1969), 1033; E. K. Nelson, J. Am. Chem. Soc. 39 (1917), 1466)

The present invention is directed to preparation of active extracts/bioactive fraction/isolate from the plant Zinziber officinale which include their chemical characterization, fingerprint profiling and methods of using such products to enhance bioavailability and/or bioefficacy of drugs, natural products and essential nutraceuticals. The present invention is directed to preparation of composite bioenhancers comprising polar and non-polar extracts of parts of Zingiber officinale and/or piperine (Ex: Piper nigrum and Piper longum) which increased significantly (25-435%), the bioavailability of a number of classes of drugs, for example, but not limited to antibiotics, antifungals, anti-virals, anticancer, cardiovascular, CNS, anti-inflammatory/anti-arthritic, anti-TB/antileprosy, anti-histaminic/respiratory disorders, corticosteroids, immunosppressants, anti-ulcer. Such extracts/bioactive fractions of Zinziber officinale either in presence or absence of piperine (Ex: Piper nigrum and Piper longum) have been found to be highly selective in their bioavailability/bioefficacy enhancing action.

There is a great interest and medical need for the improvement of bioavailability of a large number of drugs which are (a) poorly bioavailable, (b) given for long periods, and are (c) toxic and expensive. Maximizing oral bioavailability is therapeutically important because the extent of bioavailability directly influences plasma concentrations and consequently therapeutic efficacy and dose related toxic effects resulting after oral drug administration. Poorly bioavailable drugs remain sub-therapeutic because a major portion of a dose never reaches the plasma or exerts its pharmacological effect unless and until very large doses are given which may lead to serious side effects. Any significant improvement in bioavailability will result in lowering the dose or the dose frequency of that particular drug. Besides, inter-subject variability is inversely correlated with the extent of bioavailability. Therefore, low oral bioavailability leads to high variability and poor control of plasma concentration and pharmacodynamic effects. Inter-subject variability is particularly of concern for a drug with a narrow safety margin.

Incomplete oral bioavailability has various causes. These include poor dissolution or low aqueous solubility, poor intestinal membrane permeation, degradation of the drug in gastric or intestinal fluids and pre-systemic intestinal or hepatic metabolism. The normal practice to offset some of these problems has been to increase the dosage as stated earlier which has the concern toxicity of patients' non-compliance.

Many therapeutic treatments are also accompanied by loss of essential nutraceuticals in the course of therapy. The present invention improves nutritional status by increasing bioavailability/bioefficacy of various nutraceuticals also, which include metals and vitamins. The bioenhancers of the invention also have the potential to enhance the bioefficacy of a drug without influencing its plasma concentrations for various reasons, some of which, but not limited to, are described later in this invention under Section on ‘Bioavailability/Bioenhancing activity’

### OBJECTS OF THE INVENTION

The main object of the invention is to provide a active of extract and bioactive fraction obtained from Zingiber officinale.

Another object of the invention is to evaluate bioenhancing/bioavailability of Zingiber officinale extract or bioactive fraction in combination with drugs, nutrients, nutraceuticals, micronutrients and herbal drugs/products.

Still another object of the invention is to provide a bioenhancer composition comprising active principles of Zingiber officinale in combination with drugs, nutrients, nutraceuticals, micronutrients and herbal drugs/products.

Still another embodiment of the present invention is to provide a bioenhancer composition comprising extract/isolate and/or bioactive fractions obtained from Zingiber officinale, piperine and one or more selected from the group consisting of drugs, nutrients, nutraceuticals, micronutrients and herbal drugs/products.

Yet, another object of the invention is to provide a process for isolating bioactive faction from Zingiber officinale useful as a bioenhancer.

Yet another object of the invention is to provide a process for isolating bioactive faction from Zingiber officinale using aqueous and/or alcoholic solvent.

### SUMMARY OF THE INVENTION

Accordingly, the present invention provides a bioenhancing composition comprising an effective amount of an extract and/or one or more bioactive fractions/isolates of Zingiber officinale; one or more additive selected from drugs, nutrients, nutraceuticals, herbal drugs/products, micro nutrients, antioxidants and pharmaceutically acceptable additives/excipients; and optionally an effective amount of piperine or extract of piper nigrum or piper longum. The invention also provides a process for the preparation of aqueous extract, aqueous alcoholic extract and bioactive fraction from plant Zingiber officinale useful as a bioenhancer/bioavailability facilitator.

### BRIEF DESCRIPTION OF THE ACCOMPANYING DRAWINGS

FIG. 1 represents flow sheet for preparation of ginger juice, ar.extract and aq.alcoholic extract from plant Zingiber officinale

FIG. 2 represents flow sheet for fractionation of extracts of plant Zingiber officinale.

FIG. 3 represents HPLC chromatogram of dry extract Ouice) of Zingiber officinale

FIG. 4 represents HPLC chromatogram of aqueous extract of Zingiber officinale

FIG. 5 represents HPLC chromatogram of aqueous alcoholic extract of Zingiber officinale

FIG. 6 represents HPLC chromatogram of fraction 1 of aqueous alcoholic extract of Zingiber officinale

FIG. 7 represents HPLC chromatogram of fraction 2 of aqueous alcoholic extract of Zingiber officinale

FIG. 8 represents HPLC chromatogram of fraction 3 aqueous alcoholic extract of Zingiber officinale

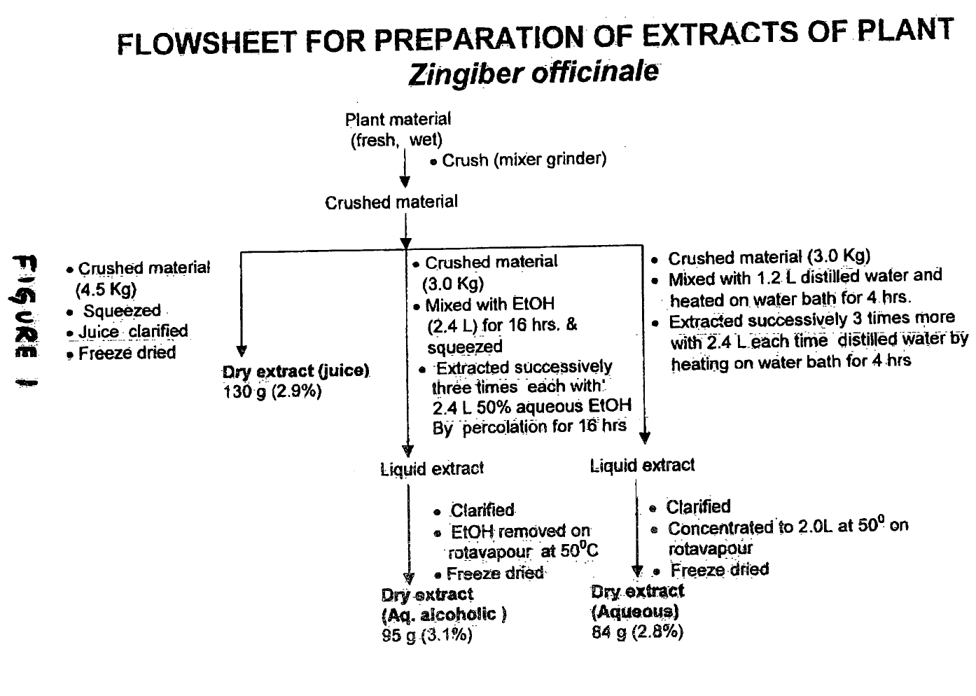

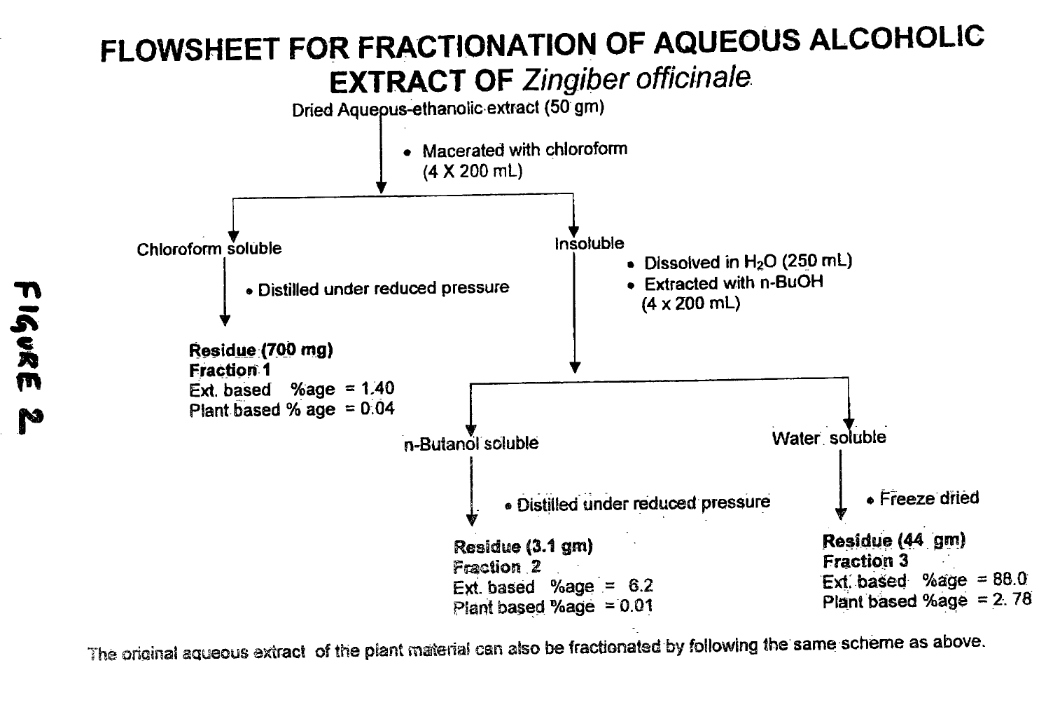

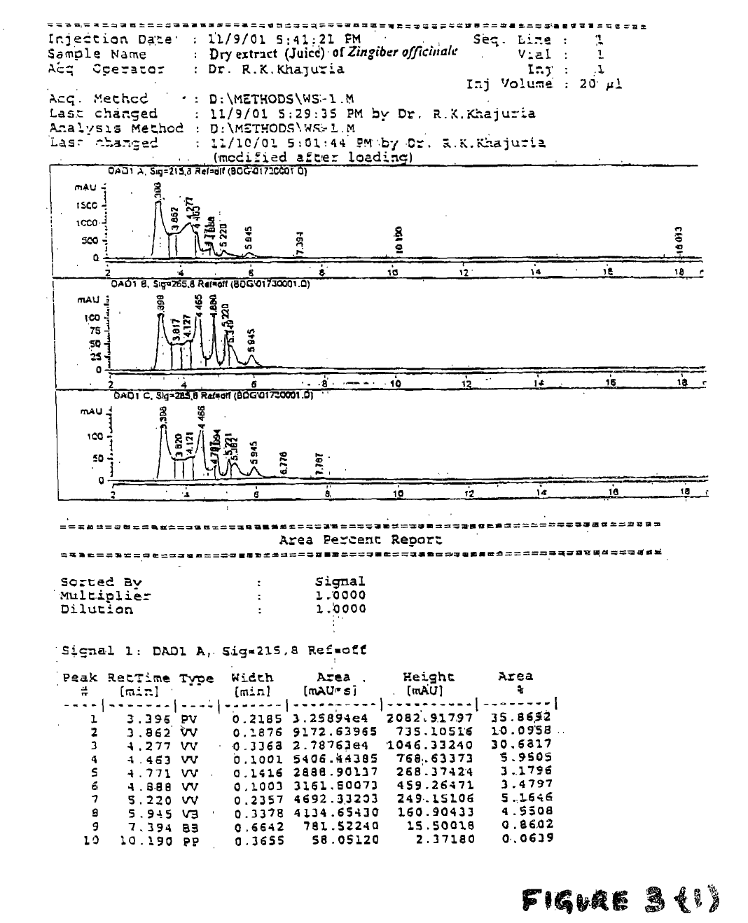

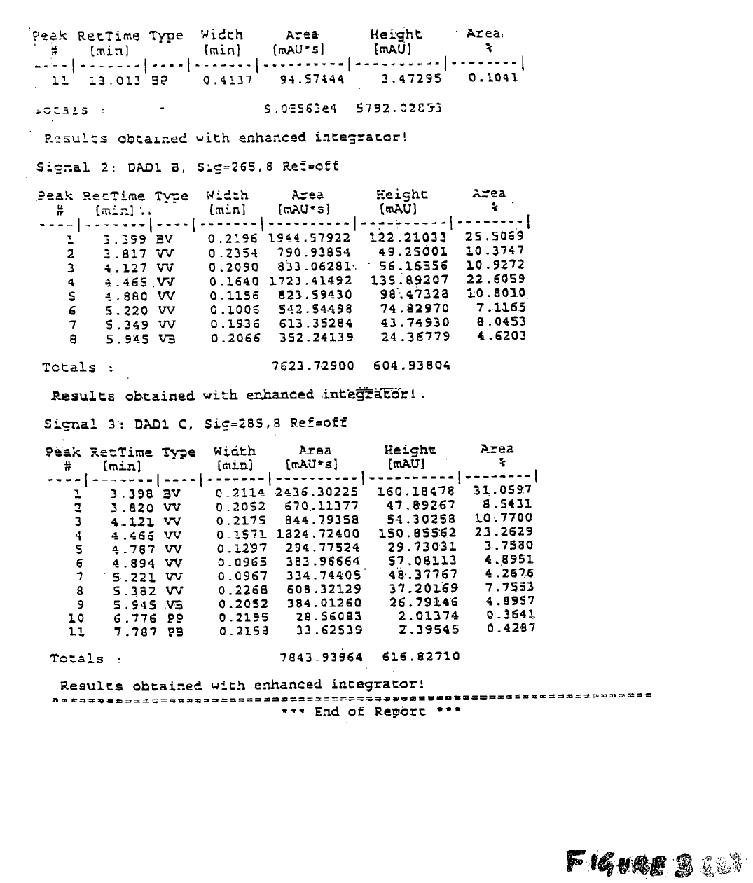

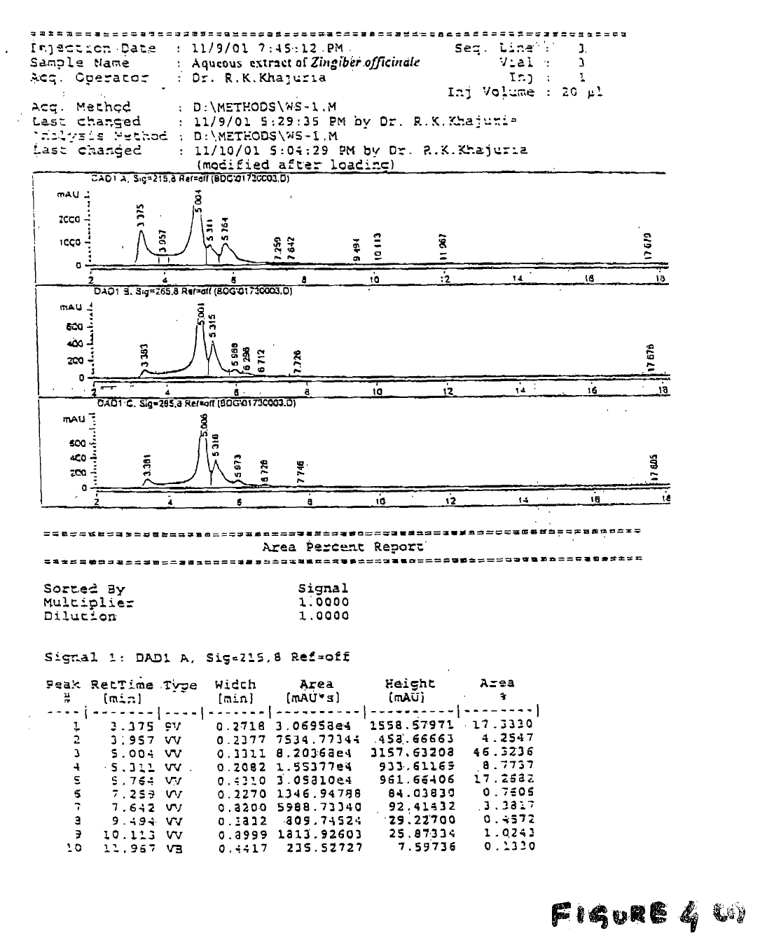

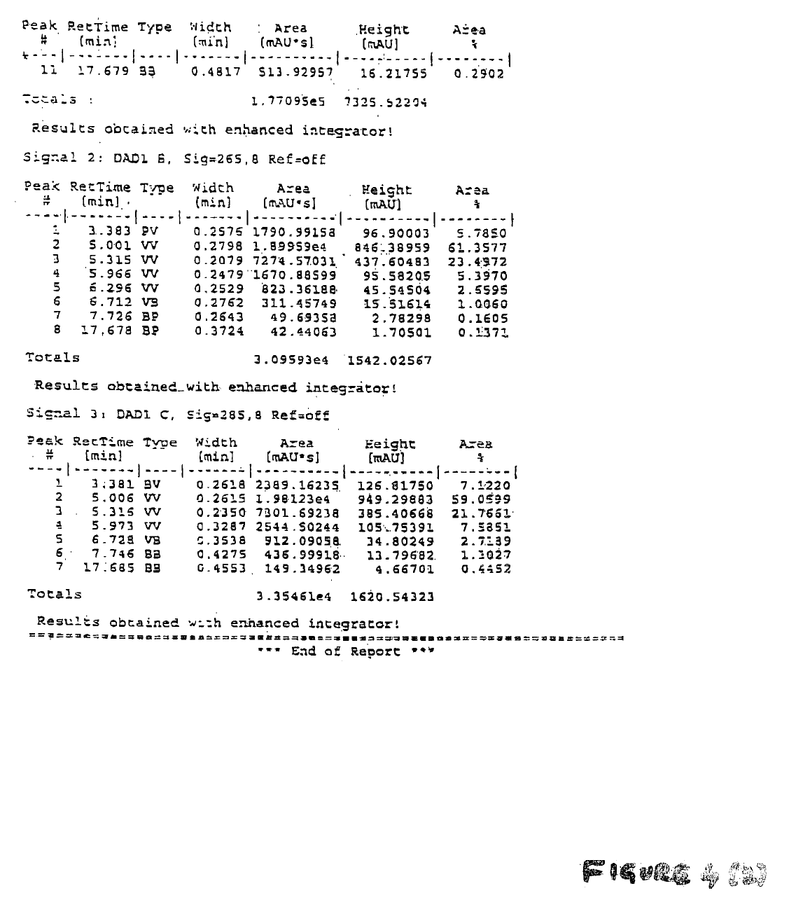

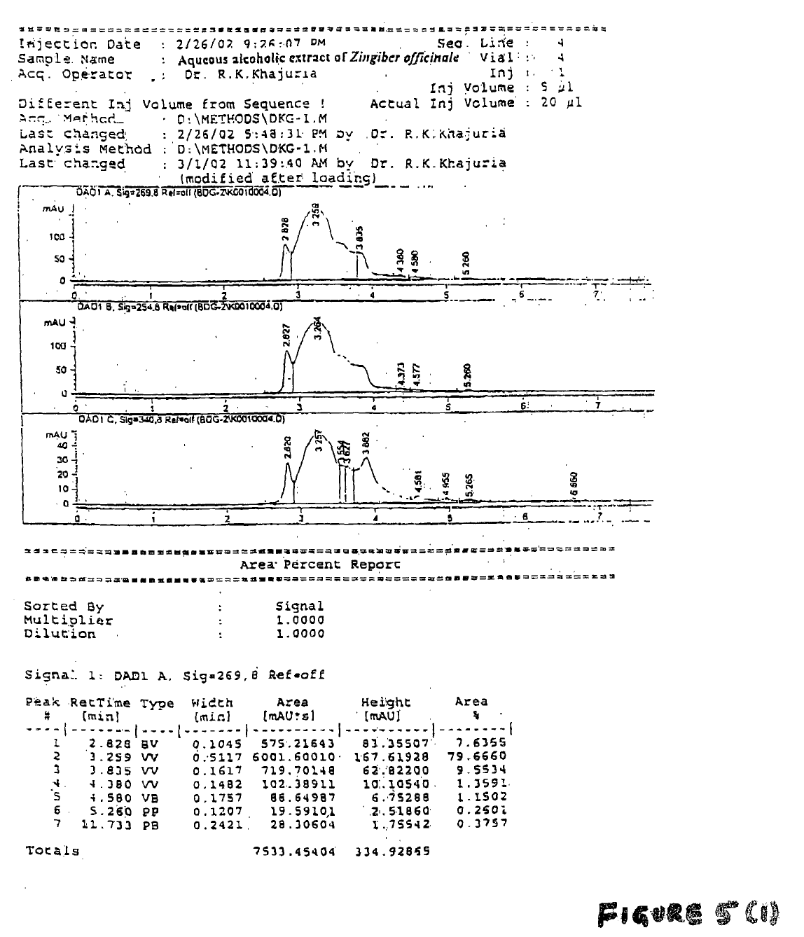

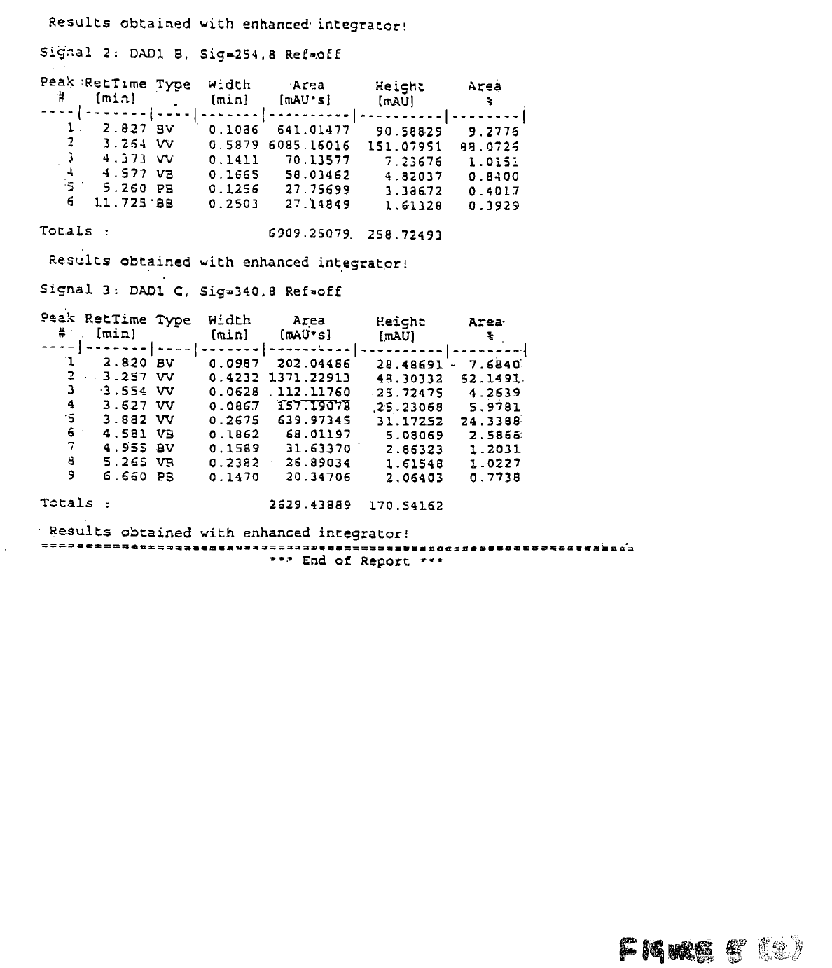

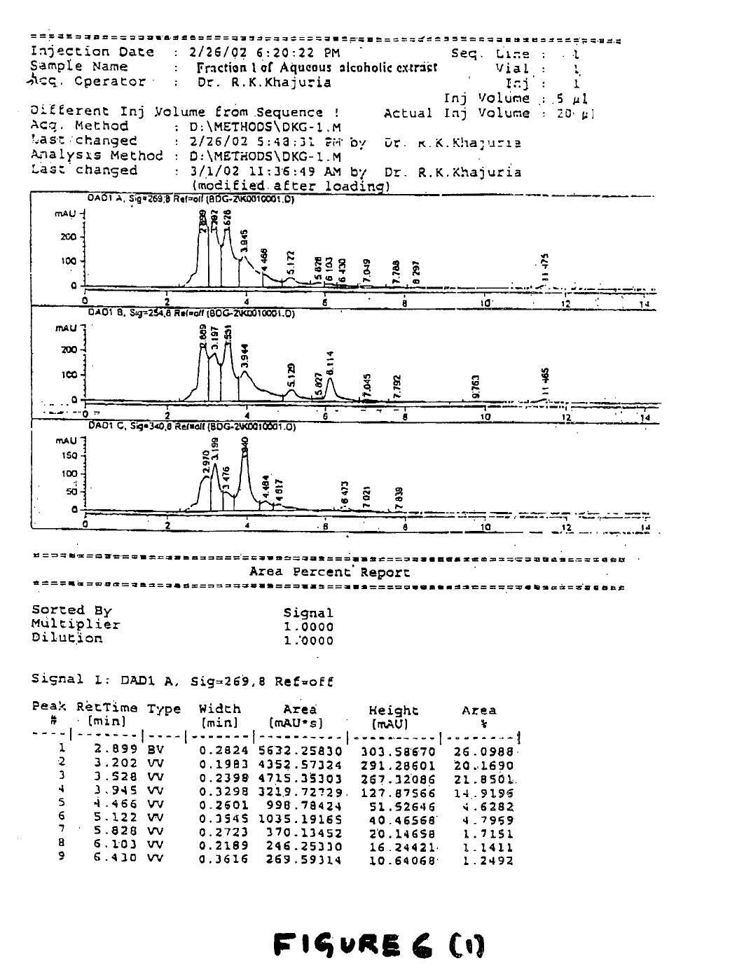

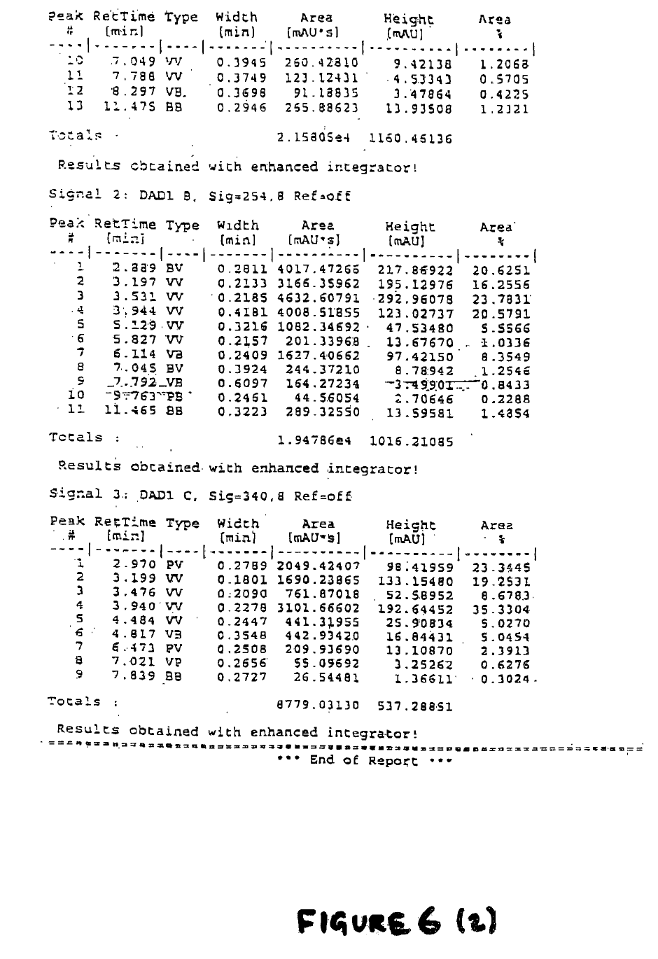

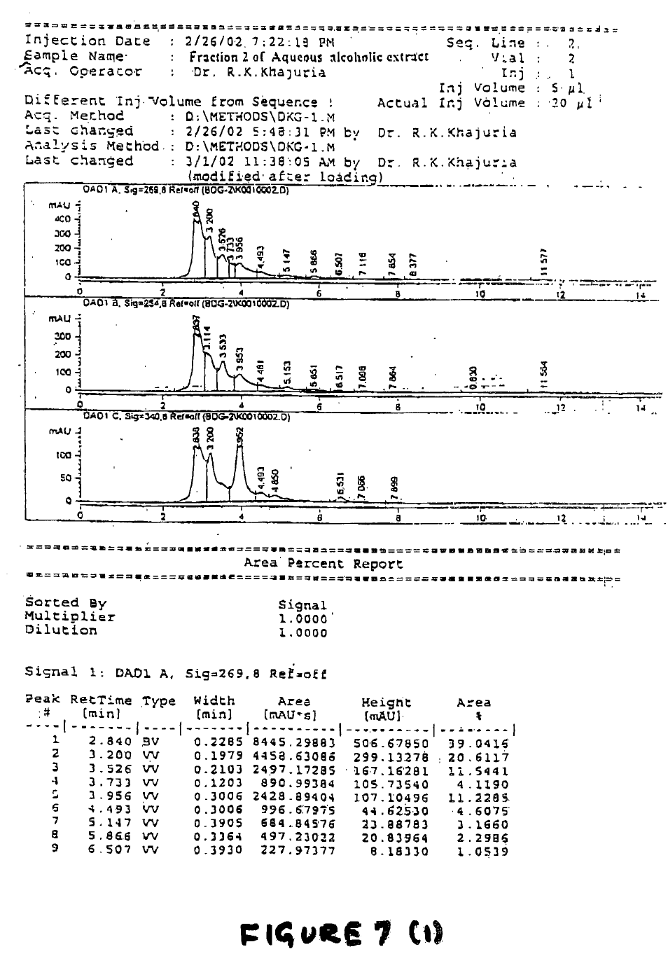

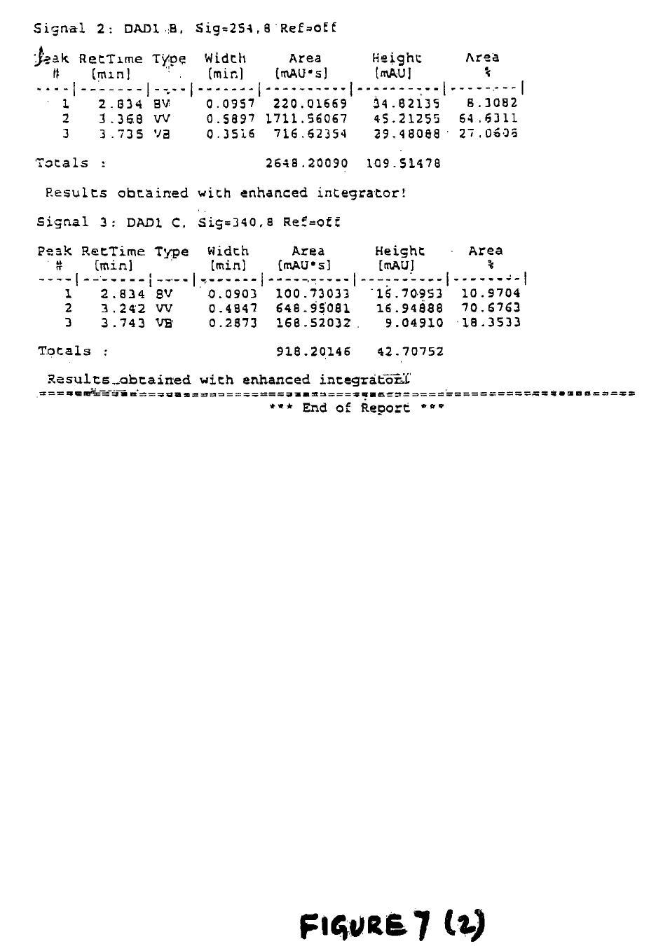

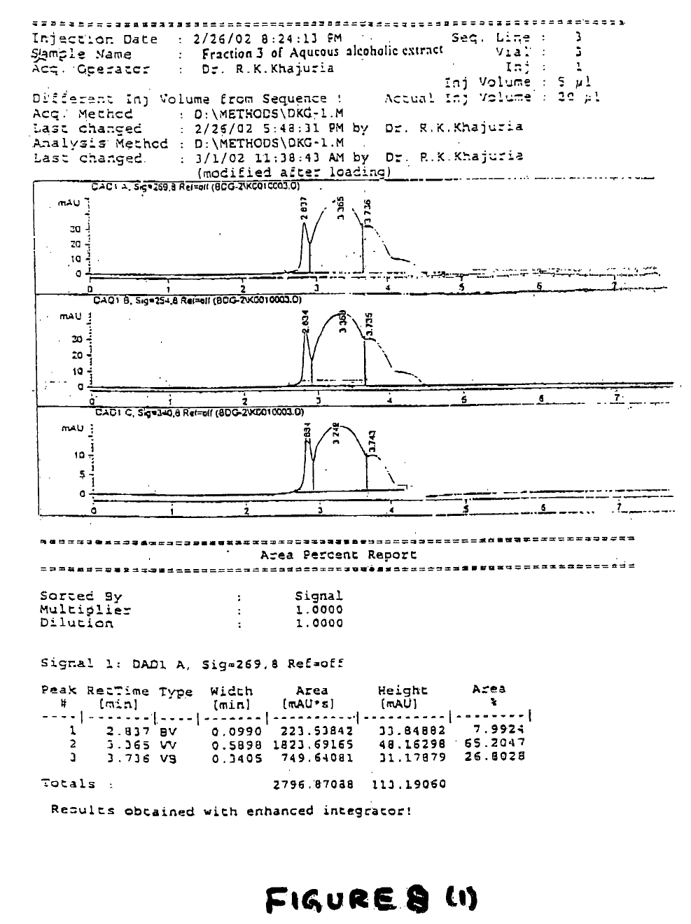

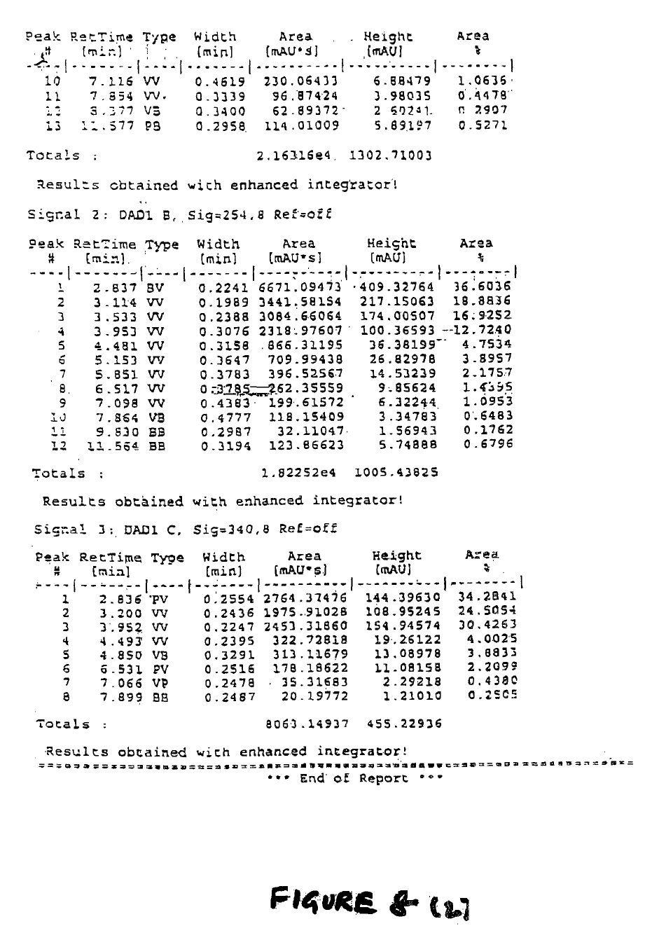

### DETAILED DESCRIPTION OF THE INVENTION

Accordingly, the present invention provides a bioenhancing composition comprising:

i. an effective amount of an extract and/or one or more bioactive fractions/isolates of Zingiber officinale;

ii. one or more additive selected from drugs, nutrients, nutraceuticals, herbal drugs/products, micro nutrients, antioxidants and pharmaceutically acceptable additives/excipients; and

iii. optionally an effective amount of piperine or extract of piper nigrum or piper longum

In an embodiment of the invention provides a composition, in which the amount of Zingiber officinale extract used is in the range of about 2.0 to 250 mg

Another embodiment provides a composition, wherein the amount of Zingiber officinale fraction/pure isolates used is in the range of about 0.5 to 75 mg

Still another embodiment provides a composition, wherein the amount of piperine used is in the range of about 5 to 50 mg.

Yet another embodiment, wherein the amount of the extract used is in the range of about 10 to 150 mg.

Yet another embodiment, wherein the amount of the fraction/pure isolates used is in the range of about 0.5 to 45 mg.

Yet another embodiment, wherein the amount of piperine used is in the range of about 3 to 15 mg.

Still another embodiment, the piperine is isolated from piper nigrum, piper longum or its oleoresin.

Another embodiment of the invention provides a composition in which the drugs used are selected from the group consisting of antibiotics, antifungal, antiviral, anticancer, cardiovascular, CNS drugs, anti-inflammatory/anti arthritic, anti-TB/antileprosy drugs, anti histamines/drugs for respiratory disorders, corticosteriods, immuno-suppressants, anti-ulcer drugs and herbal drugs.

Yet another embodiment, the antibiotic used is selected from the group consisting of quinolones, macrolides, cephalosproins, penicillins and aminoglycosides wherein quinolone is selected from the group consisting of Ciprofloxacin, Pefloxacin, Ofloxacin and Norfloxacin; the macrolide is selected from the group consisting of Erythromycin, Roxythromycin and Azithromycin; the cephalosproins is selected from the group consisting of Cefalexin, cefatrioxone, cefixime and Cefadroxil; the penicillin is selected from the group consisting of Amoxycillin and Cloxacillin; and aminoglycocide is selected from the group consisting of Amikacin and Kanamycin.

In yet another embodiment, the anti-fungal drug used is selected from the group consisting of Fluconazole, Amphotericin B and Ketoconazole.

In yet another embodiment, the antiviral drug used is selected from the group consisting of Acyclovir and Zidovudine.

In yet another embodiment, the anticancer drug is selected from the group consisting of Methotrexate, 5-Fluorouracil, Doxorubicin and Cisplatin.

In yet another embodiment, the cardiovascular drug is selected from the group consisting of Amlodipin, Lisinopril, propranolol and Atenolol.

In yet another embodiment, wherein CNS drugs is selected from the group consisting of Alprazolam and Haloperidol

In yet another embodiment, wherein anti-inflammatory/anti-arthritic drug is selected from the group consists of Diclofenac, Piroxicam, Nimesulide and Rofecoxib.

In yet another embodiment, anti-TB/anti-leprosy drug is selected from the group consisting of Rifampicin, Ethionamide, Isoniazid, Cycloserine, Pyrazinamide, Ethambutol and Dapsone

In yet another embodiment, antihistamine/drugs for respiratory disorders compound is selected from the group consisting of Salbutamol, Theophylline, Bromhexine and Loratidine

In yet another embodiment, corticosteriod is selected from the group consisting of Prednisolone, dexamethasone and Betamethasone

In yet another embodiment, immuno-supressant is selected from the group consisting of Cyclosporin A, Tacrolimus and Mycophenolatemofetil.

In yet another embodiment, wherein the anti-ulcer compound is selected from the group consists of Rantidine, Cimetidine and Omerprazole.

In another embodiment, the herbal product/drug is selected from Echinacea, Tinospora cordifolia, Picrorrhiza kurroa, Aegles marmelos, Andrographis paniculata, Emblica ribes, Asparagus racemosus, Terminalia chebula Withania somnifera, Centella asiatica and/or their mixture thereof.

In yet another embodiment, wherein the nutrient is selected from group consists of sugar, carbohydrates, fats and proteins.

One more embodiment of the present invention provides a composition, wherein vitamin used is selected from the group consisting of Vitamin A, Vitamin E, Vitamin B1, Vitamin B6, Vitamin B12, Vitamin C and Folic acid.

The antioxidant used for preparing the bioenhancing composition is selected from the group consisting of β-Carotene, Silymarin, Selenium, Lycopene and Ellagiogallotannins

The natural herbal product used is selected from the group consisting of Curcumin, Boswellic acids and Ruti n and essential micro nutrients is selected from the group consisting of Methionine, Lysine, Leucine, Valine, Isoleucine, Zinc, Calcium, Glucose, Potassium, Copper and Iron

In yet another embodiment of the invention, the plant extract of Zingiber officinale or its fraction/pure isolate used is extracted from any plant parts of Zingiber officinale

One more embodiment of the invention related to administration of the bioenhancing composition. The composition is administered through oral, parenteral, nasal, inhalation including nebulisers, rectal, vaginal, transdermal and any others suitable routes.

In yet another embodiment, the bioenhancing effect of the extracts/fractions/pure isolates of Zingiber officinale alone or in combination with piperine is selective in enhancing the bioavailability/bioefficacy of a drug, nutraceutical, and herbal drug/formulation.

In another embodiment of the invention, wherein bioavailability/bio-enhancing activity provided by Zingiber officinale alone is up to 75%

Yet another embodiment, the composition containing Zingiber officinale alone provides bioavailability/bio-enhancing activity in the range of 30-75%

One more embodiment of the invention provides a composition, wherein the composition containing piperine and Zingiber officinale, further enhances the bioavailability of drugs in the range of 10 to 85% beyond Zingiber officinale alone.

Another embodiment, the dosage of bioehancer from Zingiber officinale as extract is in the range of 10 to 30-mg/kg/body weight and piperine is in the range of 4 to 12 mg/kg/body weight.

Another embodiment, the dosage of bioehancer from Zingiber officinale as bioactive fraction is in the range of 5 to 15-mg/kg/body weight, preferably 30-mg/kg/body weight, and piperine is in the range of 6 to 10 mg/kg/body weight, preferably 8-mg/kg/body weight.

One more embodiment of the present invention provides a process for the preparation of an aqueous extract, aqueous alcoholic extract and bioactive fraction from the plant Zingiber officinale, said process comprises steps of:

a) extracting crushed plant material with water or aqueous alcoholic solvent at a temperature range of 95-100° C.;

b) cooling and filtering the extract of step (a) to obtain a clear aqueous extract or aqueous alcoholic extract;

c) evaporating the aqueous extract of step (b) under reduced pressure at 60° C. to obtain an concentrated aqueous extract;

d) freeze drying the concentrated aqueous extract of step (c) to obtain a dried aqueous extract;

e) evaporating the solvent from aqueous alcoholic extract of step (b);

f) macerating dried aqueous alocholic extract obtained from step (e) of with chloroform,

g) separating the chloroform soluble fraction from step (f) to obtain fraction 1 and an insoluble fraction;

h) dissolving the insoluble fraction from step (g) in water and adding alcoholic solvent, stirring and filtering the mixture to separate aqueous alcohol soluble portion and a residue;

i) distilling the aqueous alcohol soluble fraction of step (h) under reduced pressure to obtain fraction 2; and

j) dissolving the residue of step (i) in water and freeze drying it to get fraction 3.

Another embodiment of the invention, wherein the solvents used are selected from aqueous, aqueous-alcoholic, ketonic, etheral and halogenated solvents.

Still another embodiment of the invention, wherein the alcoholic solvent used is selected from the group consisting of methanol, ethanol, propanol and/or aqueous alcoholic solvent.

Still another embodiment of the invention, the ratio of plant material used in step (a) to the solvent used is in the range of 1:1 to 1:3, preferably 1:2.5.

Bioavailability/bioefficacy enhancing activity

The aqueous, aqueous—alcoholic, ketonic, ethereal, halogenated solvents extracts of the plant parts were evaluated with different therapeutic categories of drugs and nutrients (vital amino acids, metals, antioxidants, vitamins) and herbal drugs. The bioavailability/bioefficacy enhancing (BE) activity of the extracts was found to be consistent from 10 mg to 150 mg irrespective of the amount of the drug(s) present in the formulation. Sub-bioactive fractions of the active extracts were also evaluated, with the same categories of drugs. The BE activity of the bioactive fraction (s) increased corresponding to their proportions in the parent extract. The doses of the fraction (s) responsible for the BE activity ranged from 0.5 to 45 mg. The parent extract as well as the active fraction (s) were found to be active individually as well as in combination with each other with different categories of drugs. The bioenhancer activity of the fraction (s) was found to be consistent from 2.0 mg to 30.0 mg irrespective of the amount of the drug (s) present in the formulation. The BE activity of the fraction (s) was more enhanced as compared to that of the parent extracts.

The extracts or its bioactive fractions were found to be 25-80% more active when used individually in combination with piperine (1-piperoyl piperidine). Besides both the parent extracts as wells as their bioactive fractions in different combinations showed pronounced activity ranging from 20-70% in presence of piperine. The amount of piperine in these formulations ranging from 03-15 mg.

The extracts or its fractions either in presence or absence of piperine have been found to be highly selective in their bioavailability enhancing activity. This is apparent from the degree of bioavailibility enhancement caused by these extracts/bioactive fractions. It varies from nil to nearly significant (15%) to highly significant (120%). The reasons for this rather selective pattern as applicable to formulations with or without piperine may be as follows:

The extract or its bioactive fraction (s) have been found to be highly selective in their bioavailability enhancing activity. This is more than apparent from the degree of bioavailability enhancement caused by these extract/fraction (s). It varies from almost nearly significant (20%) to highly significant (200%). The reasons for this non-uniform or rather selective pattern as applicable to formulations with or without piperine may be as follows:

1. The extracts/fractions may be enhancing the absorption/transport of certain drugs/nutrients from the gastrointestinal tract.

2. They may be inhibiting partially the specific drug metabolising enzymes, responsible for selective biotransformation of molecules, thus prolonging the elimination or biological half-life of the drug.

3. An increased penetration of therapeutic drugs into their cellular/molecular targets could also be one of the reasons.

4. The formulations may also affect the protein/tissue binding of active drugs, which may be responsible for enhanced bioenhancing effect.

5. Direct potentiation of mechanism of action of a drug may be an important factor contributing to enhanced bioavailability.

6. An enhanced immune response of the host by the incorporation of bioenhancer may cause increase in therapeutic response of the active drugs.

7. A combination of either two or more than two factors as enumerated above [Ser. No. 1-6] may be prevalent in the overall bioenhancing effect.

8. The reasons for this selective pattern may be attributable to one or more than one of the following factors: (a) promoting the absorption of drugs from GIT (b) inhibiting/reducing the rate of biotransformation of drugs in the liver or intestines (c) modifying the immune system in a way that the overall requirement of the drug is reduced substantially (d) increasing the penetration or the entry into the pathogens even where they become persistors within the macrophages such as for Mycobacterium tuberculosis and such others. This eventually ensures the enhanced killing of these organisms well secured within the places otherwise inaccessible to the active drug, (e) Inhibiting the capability of pathogens or abnormal tissue to reject the drug e.g., efflux mechanisms frequently encountered with anti-malarial, anti-cancer and anti-microbial drugs (f) modifying the signaling process between host and pathogens ensuring increased accessibility of the drugsto the pathogens (g) Enhancing the binding of the drug with the target sites such as receptors, proteins, DNA, RNA and the like in the pathogen, thus potentiating and prolonging its effect leading to enhanced antibiotic activity against pathogens (h) Besides above plausible modes of action the bioenhancer agents may also be useful for promoting the transport of nutrients and the drugs across the blood- brain barrier which could be of immense help in the control of diseases like cerebral infections, epilepsy and other CNS problems.

Primarily, but not exclusively, the invention enhances the carrier-mediated entry of the drug and also thepassive diffusion and the active transport pathways in the tissue which are responsible for transporting physiological substances such as nutraceutical to their target sites. As applicable to any mechanism of action the products of this invention contribute in a synergistic and/or additive manner so that most drugs and nutraceuticals in presence of the products described in the present art are more bioavailable or bioefficaceous as a result of one or more of the mechanisms. The bioavailability and the bioefficacy of drugs and nutraceuticals is also relevant to animal health besides being important for humans. The invention therefore is also intended to be used in veterinary preparations.

The invention further relates to the isolation of an extract and/or its fraction from the plant Zingiber officinale, its standardization with its intended use as drug bioavailability enhancer for the drugs belonging to therapeutic categories such as antimicrobial, antifungal, anti-viral, antitubercular, antileprosy, antiinflammatory, antiarthritic, cardiovascular, antihistaminics, respiratory distree relieving drugs, immunosuppressants, nutraceuticals in compositions to be administered orally/parenterally, topically, inhalations (including nebulizers), rectally, vaginally in human beings and/or veterinary conditions.

In addition, the invention relates to the preparation of a formulation containing extract and/or its fraction/isolate from the plant Zingiber officinale, and piperine, its standardization with its intended use as drug bioavailability enhancer for the drugs belonging to therapeutic categories such as antimicrobial, antifungal, anti-viral, antitubercular, antileprosy, antiinflammatory, antiarthritic, cardiovascular, antihistaminics, respiratory distress relieving drugs, immunosuppressants, nutraceuticals in compositions to be administered orally/parenterally, topically, inhalations (including nebulizers), rectally, vaginally in human beings and/or veterinary conditions

The bioavailability enhancer principle may be any extract, its bioactive fraction and/or a pure isolate from the plant.

The bioavailability enhancer principle may be any extract, its bioactive fraction and/or a pure isolate of the plant in combination with piperine

A process for the preparation of extract (s)/bioactive fractions(s)/pure isolate (s) which may involve the use of water, alcohol, combinations of water and alcohol, halogenated hydrocarbons, ketones, ethers as solvents.

A process for the preparation of extract (s)/bioactive fractions(s)/pure isolate (s) having piperine which may involve the use of water, alcohol, combinations of water and alcohol, halogenated hydrocarbons, ketones, ethers as solvents

A process for preparation of bioactive fractions/pure isolates with or without piperine making use of physical techniques like dialysis/molecular sieves/membranes, variety of chromatographic techniques and/or liquid-liquid or solid phase extractions, followed by their complete finger print profiles (HPLC/HPTLC/LC-MS-MS)

The formulation of a drug selected from any of the therapeutic categories of the drugs, nutraceuticals, herbal drugs/formulations in combination with the bioenhancer may be intended for routes of administration viz., oral, parenteral, nasal, inhalation including nebulisers, rectal, vaginal, transdermal and others.

The bioenhancing effect of the extracts/bioactive fractions/pure isolates of Zingiber officinale either alone or in combination with piperine is selective and does not enhance the bioavailability/bioefficacy of each and every drug, nutraceutical, herbal drug/formulation.

The amount of the extracts in the bioavailablity/bioefficacy enhanced formulation (s) may range from 05 to 75 mg irrespective of the amount of drugs in the formulation (s).

The amount of the fraction/pure isolate in the bioavailability/bioefficacy enhanced formulation (s) may range from 1.0 to 30.0 mg irrespective of the amount of drug (s) incorporated in the formulation (s).

That the extracts/fractions/pure isolates or piperine express no biological or toxicological effect of their own at the doses at which they are intended The following examples are intended to demonstrate some of the preferred embodiments and in no way should be construed to limit the scope of the invention. Any person skilled in the art can design more formulations, which may be considered as part of the present invention.

### EXAMPLES

### Example 1

Preparation of colourless, non-pungent piperine by a novel process. Commercially available Piper nigrum or Piper longum or their oleoresins have been used as the source material. 20 kg long pepper oleoresin is extracted with chlorinated solvents like CHCl₃, CH₂Cl₂, C₂H₄Cl₂ (25 litre) for six hours or 20 kg black pepper powder is soxhletted with toluene for 8 hours. The extracts are concentrated to dryness under reduced pressure and dissolved in ethanol at 78° C. The ethanolic solution is adsorbed over neutral Al₂O₃ and packed in a glass column. Elution is carried out with CHCl₃:EtOH (9:1) and the eluate is concentrated to dryness and dissolved in minimum quantity of ethanol. The solution is treated with activated charcoal and filtered through a celite bed. The filtrate is concentrated to saturation point, cooled when colorless crystalline precipitate is obtained. The precipitate is separated by suction filtration and dried.

The specifications of the preferred materials are as under:

Colour: Colourless monoclinic prismatic crystals; Melting point: 131° C.;

Assay: Minimum (99.1% (by LC/MS)

### Example 2

The extract and the active fractions are prepared from the plant material Z. officinalis as per the flow chart accompanying the specification.

Preparation and fully finger printed (HPLC) profile of the products is appended separately.

### Example 3

Doses, models/design of experiment and estimation methodology in a typical experiment

Note

1. Bioenhancers (BE) from Zingiber officinalis means either the aqueous, or 50% alcoholic extract or fraction No. 1.

2. In case of Zingiber officinale, not withstanding the difference in dose of extract or its fraction, the enhancement caused in the bioavailability of the drug with which they are combined remains the same, because the dose of the fraction used is proportionate to its concentration in the extract.

3. The doses remained unchanged even when the bioenhancers (BEs) were used in combination with each other.

### Example 4

### Doses

(i) Bioenhancer (BE) from Zingiber officinale

Extract: 30 mg/kg body weight (Rats)

Fraction No 1: 15 mg/kg body weight (Rats)

(ii) Piperine: 8 mg/kg body weight (Rats)

(iii) Bioenhancer from Zingiber officinale: 35 mg/kg body weight (Rats)

As an example of an experiment in Rat (fasted):

Drug: Rifampicin, 40 mg/kg

BE (Zingiber officinale & others): Doses as in Example No. 4

Experimental procedure: Drug alone/or in combination with BE was administered to rats as per the following design:

Group 1: Control

Group 2: Rifampicin alone

Group 3: BE alone

Group 4: Rifampicin+BE (Zingiber officinale)

Blood from control/treated animals at predetermined intervals (0-24 hrs) (Total 14 timings) Rifampicin was extracted from the blood (plasma) using dichloromethane. The concentration of rifampicin in the samples was determined using HPLC (Model: Shimadzu 1080 BP); PDA detector; Mobile phase: phosphate buffer: acetonitile (40:60); Flow rate 1.0 ml/min. Column RP 18.

Control and BE only groups were employed to study the interference of plasma component and the bioenhancer used.

### Example 5

The above methodology was adapted for evaluating the bio-enhancing activity of other drugs, micro nutrients, nutracuticals, nutrients and other herbal products and the enhancing effects are tabulated under each heading.

List of drugs, nutraceuticals, herbal formulations cited below as some of the example for the purpose of present invention. The following tables illustrates the bio enhancing activity of Zingiber officinale extract and/or active fraction, Piperine and combination of Piperine+Zingiber officinale extract and/or active fraction.

A. Drugs

| | Categories | Drugs |
|---|---|---|
| I | Antibiotics | Fluoroquinolones |
| | | Cipro-, Nor-, P-, and 0-floxacins Macrolides |
| | | Erythro-, Roxythro-, and Azithromycin |
| | | Cephalosporins |
| | | Cefalexin, Cefadroxil, cefatrioxone and |
| | | Cefixime |
| | | Penicillins |
| | | Amoxycillin, Cloxacillin |
| | | Aminoglycosides |
| | | Amikacin, Kanamycin |
| II. | Antifungal | Fluconazole, Amphotericin B, |
| | | Ketoconazole |
| III. | Anti-viral | Acyclovir, |
| | | Zidovudine |
| IV. | Anti-cancer | Methotrexate |
| | | 5-Fluorouracil |
| | | Doxorubicin |
| | | Cisplatin |
| V. | Cardiovascular | Amlodipin |
| | drug | Lisinopril |
| | | Atenolol |
| VI. | CNS drugs | Alprazolam |
| | | Haloperidol |
| VI. | Anti- | Diclofenac |
| | inflammatory/ | Piroxicam, |
| | antiarthritic | Nimesulide |
| | (NSAID) | Rofecoxib |
| VII. | Anti-TB/ | Rifampicin Ethionamide |
| | Antileprosy drugs | Isoniazid Cycloserine |
| | | Pyrazinamide |
| | | Ethambutol |
| | | Dapsone |
| VIII. | Anti histamines/ | Salbutamol |
| | respiratory | Theophylline |
| | disorders | Bromhexine, Loratidine |
| IX | Corticosteroids | Prednisolone, dexamethasone, Betamethasone |
| X. | Immuno- | Cyclosporin A, Tacrolimus |
| | suppressants | Mycophenolate mofetil |

Drugs Category:

I. Antibiotics:

| | % Enhancement in bioavailability | | |
|---|---|---|---|
| | BE from | Piperine | Piperine + BE from |
| Drug | Zingiber officinale | as BE | Zingiber officinale |

(a) Fluroquinolones

| | | | |
|---|---|---|---|
| Ciprofloxacin | 68 | 55 | 70 |
| P-floxacin | 53 | 61 | 69 |
| O-floxacin | 49 | 52 | 67 |
| Norfloxacin | Negligible | Negligible | Negligible |
| (b) Macrolides | | | |
| Erythromycin | 68 | 95 | 105 |
| Roxythromycin | 72 | 110 | 93 |
| Azithromycin | 78 | 89 | 85 |

(c) Cephalosporins

| | | | |
|---|---|---|---|
| Cefalexin | 75 | 90 | 85 |
| Cefadroxil | 68 | 70 | 65 |
| Cefatrioxone | Nil | Nil | Nil |
| Cefixime | Nil | Nil | Nil |
| (d) Penicillins | | | |
| Amoxycillin | 80 | 120 | 90 |
| Cloxacillin | 76 | 87 | 90 |

(e) Aminoglycosides:

| | | | |
|---|---|---|---|
| Amikacin | Nil | Negligible | Nil |
| Kanamycin | 65 | 72 | 92 |

II. Antifungal

| | % Enhancement in bioavailability | | |
|---|---|---|---|
| | BE from | Piperine | Piperine + BE from |
| Drug | Zingiber officinale | as BE | Zingiber officinale |
| Fluconazole | 120 | 87 | 110 |
| Amphotericin B | Nil | Negligible | negligible |
| Ketoconazole | 125 | 105 | 100 |

III. Anti-viral

| | % Enhancement in bioavailability | | |
|---|---|---|---|
| | BE from | | Piperine + BE from |
| Drug | Zingiber officinale | Piperine as BE | Zingiber officinale |
| Acyclovir | 82 | 96 | 85 |
| Zidovudine | 105 | 140 | 126 |

IV. CNS drugs:

| | % Enhancement in bioavailability | | |
|---|---|---|---|
| | BE from | | Piperine + BE from |
| Drug | Zingiber officinale | Piperine as BE | Zingiber officinale |
| Aiprazolam | 76 | 65 | 70 |
| Halopendol | Nil | Nil | Nil |

V. Anti-cancer

| | % Enhancement in bioavailability | | |
|---|---|---|---|
| | BE from | Piperine | Piperine + BE from |
| Drug | Zingiber officinale | as BE | Zingiber officinale. |
| Methotrexate | 87 | 65 | 49 |
| 5-Fluorouracil | 110 | 87 | 93 |
| Doxorubicin | 72 | 68 | 75 |
| Cisplatin | 56 | Negligible | 45 |

VI. Cardiovascular:

| | % Enhancement in bioavailability | | |
|---|---|---|---|
| | BE from | | Piperine + BE from |
| Drug | Zingiber officinale | Piperine as BE | Zingiber officinale |
| Amlodipine | 68 | 43 | 95 |
| Lisinopril | 76 | 85 | 100 |
| Atenolol | Nil | Negligible | Negligible |
| Propranolol | 76 | 84 | 104 |

VII. Anti-inflammatory/antiarthritic:

| | % Enhancement in bioavailability | | |
|---|---|---|---|
| | BE from | | Piperine + BE from |
| Drug | Zingiber officinale | Piperine as BE | Zingiber officinale. |
| Diclofenac | 90 | 120 | 140 |
| Piroxicam | 86 | 110 | 134 |
| Nimesulide | 144 | 132 | 165 |
| Rofecoxib | Negligible | Negligible | Negligible |

VIII. Anti-TB/Antileprosy drugs:

| | % Enhancement in bioavailability | | |
|---|---|---|---|
| | BE from | Piperine | Piperine + BE from |
| Drug | Zingiber officinale | as BE | Zingiber officinale. |
| Rifampicin | 65 | 45 | 98 |
| Isoniazid | Nil | nil | Negligible |
| Pyrazinamide | Nil | nil | Nil |
| Ethambutol | Nil | Nil | Nil |
| Dapsone | 46 | 34 | 55 |
| Ethionamide | 56 | 45 | 48 |
| Cycloserine | 71 | 67 | 70 |

IX. Anti-histamines/respiratory disorders:

| | % Enhancement in bioavailability | | |
|---|---|---|---|
| | BE from | | Piperine + BE from |
| Drug | Zingiber officinale. | Piperine as BE | Zingiber officinale |
| Salbutamol | 78 | 60 | 92 |
| Theophylline | 76 | 65 | 80 |
| Bromhexine | 46 | 67 | 75 |
| Loratidine | Nil | Nil | Nil |

X. Corticosteroids:

| | % Enhancement in bioavailability | | |
|---|---|---|---|
| | BE from | Piperine | Piperine + BE from |
| Drug | Zingiber officinale. | as BE | Zingiber officinale |
| Prednisolone | Nil | Nil | Nil |
| Dexamethasone | 76 | 66 | 80 |
| Betamethasone | 75 | 72 | 70 |

XI. Immunosuppressants:

| | % Enhancement in bioavailability | | |
|---|---|---|---|
| | BE from | Piperine | Piperine + BE from |
| Drug | Zingiber officinale | as BE | Zingiber officinale |
| Cyclosporin A | 116 | 86 | 120 |
| Tacrolimus | 75 | 105 | 117 |
| Mycophenolate | Nil | Nil | Nil |
| Mofeit | | | |

XII. Anti-ulcer

| | % Enhancement in bioavailability. | | |
|---|---|---|---|
| | BE from | | Piperine + BE |
| Drug | Zingiber officinale. | Piperine as BE | Zingiber officinale. |
| Ranitidine | 147 | 21 | 165 |
| Cimetidine | 98 | Nil | 76 |
| Omeprazole | Nil | Nil | Nil |

D. Herbal formulations:

| | % Enhancement in bioavailability/ bioefficacy | | |
|---|---|---|---|
| | BE from | Piperine | Piperine + BE from |
| Drug | Zingiber officinale. | as BE | Zingiber officinale. |
| Echinacea | 66 | 75 | 82 |
| Tinospora | 67 | 85 | 112 |
| cordifolia | | | |
| Picrorrhiza | 56 | 78 | 87 |
| kurroa | | | |
| Aegles marmelos | Nil | Nil | Nil |
| Andrographis | 55 | 63 | 70 |
| paniculata | | | |
| Emblica ribes | 65 | Nil | 72 |
| Asparagus | 44 | 58 | 60 |
| racemosus | | | |
| Terminalia | Nil | Nil | Nil |
| chebula | | | |
| Withania | 64 | 55 | 48 |
| somnifera | | | |
| Centella | Nil | Nil | Nil |
| asiatica | | | |

Nutraceutical

| | | | % Enhancement in bioavailability | | |
|---|---|---|---|---|---|
| | | | BE from | | Piperine + BE |
| | | Dose | Zingiber | | from Z. |
| | Category | (mg/kg) | officinalis | Piperine | officinalis |
| I | Vitamins | | | | |
| | Vit A | 1 | 30 | 14 | 40 |
| | Vit E | 40 | 27 | nil | 25 |
| | Vit B1 | 10 | nil | 16 | 15 |
| | Vit B6 | 0.5 | nil | nil | nil |
| | Vit B12 | 0.1 ug | nil | nil | nil |
| | Vit C | 50 | 25 | nil | 24 |
| | Folic acid | 50 ug | 34 | nil | 35 |
| II | Antioxidants | | | | |
| | B-carotene | 15 | 36 | 34 | 55 |
| | Silymarin | 5 | 28 | 13 | 43 |
| | Selenium | 2 | nil | nil | nil |
| III | Natural herbal | | | | |
| | Products | | | | |
| | Curcumin | 50 | 43 | 33 | 70 |
| | Boswellic acid | 50 | nil | nil | nil |
| | Rutin | 40 | 21 | 66 | 78 |
| IV | Essential nutritional | | | | |
| | components | | | | |
| | Methionine | 20 | 25 | 23 | 45 |
| | Lysine | 40 | 15 | 31 | 40 |
| | Leucine | 50 | 17 | 25 | 34 |
| | Valine | 25 | 25 | 26 | 46 |
| | Isoleucine | 25 | 31 | 18 | 35 |
| | Zinc | 0.1 | 19 | nil | 20 |
| | Calcium | 30 | nil | neg- | nil |
| | | | | ligible | |
| | Glucose | 50 | nil | 29 | 25 |
| | Potassium | 25 | 21 | nil | 19 |
| | Copper | 30 | nil | nil | nil |
| | Iron | 0.5 | nil | nil | nil |

1. A bioenhancing composition comprising:
i. an effective amount of an extract and/or one or more bioactive fractions/isolates of Zingiber officinale;
ii. one or more additive selected from drugs, nutrients, nutraceuticals, herbal drugs/products, micro nutrients, antioxidants and pharmaceutically acceptable additives/excipient, and
iii. optionally, an effective amount of piperine or extract/fraction of piper nigrum or piper longum.

2. The composition as claimed in claim 1, wherein the amount of Zingiber officinale extract used is in the range of about 2.0 to 250 mg.

3. The composition as claimed in claims 1, wherein the amount of Zingiber officinale fraction/pure isolates used is in the range of about 0.5 to 75 mg irrespective of the amount of drug in the composition, preferably in the range of 1 to 30 mg irrespective to the amount of the drugs in the composition.

4. The composition as claimed in claim 1, wherein the amount of piperine used is in the range of about 5 to 50 mg.

5. The composition as claimed in claim 1, wherein piperine is isolated from piper nigrum, piper longum or its oleoresin.

6. The composition as claimed in claim 1, wherein the drugs are selected from the group consisting of antibiotics, antifungal, antiviral, anticancer, cardiovascular, CNS drugs, anti-inflammatory/anti-arthritic, anti-TB/anti-leprosy drugs, anti-histamines/drugs for respiratory disorders, corticosteriods, immuno-suppressants, anti-ulcer drugs and herbal drugs.

7. The composition as claimed in claim 6, wherein the antibiotic used is selected from the group consisting of quinolones, macrolides, cephalosproins, penicillins and aminoglycosides.

8. The composition as claimed in claim 7, wherein the quinolone is selected from the group consisting of Ciprofloxacin, Pefloxacin, Ofloxacin and Norfloxacin.

9. The composition as claimed in claim 7, wherein the macrolide is selected from the group consisting of Erythromycin, Roxythromycin and Azithromycin.

10. The composition as claimed in claim 7, wherein the cephalosproins is selected from the group consisting of Cefalexin, cefatrioxone, cefixime and Cefadroxil.

11. The composition as claimed in claim 7, wherein the penicillin is selected from the group consisting of Amoxycillin and Cloxacillin.

12. The composition as claimed in calim 7, wherein the aminoglycocide is selected from the group consisting of Amikacin and Kanamycin.

13. The composition as claimed in claim 6, wherein anti-fungal drug is selected from the group consisting of Fluconazole, Amphotericin B and Ketoconazole.

14. The composition as claimed in claim 6, wherein antiviral drug is selected from the group consisting of Acyclovir and Zidovudine.

15. The composition as claimed in claim 6, wherein anticancer drug is selected from the group consisting of Methotrexate, 5-Fluorouracil, Doxorubicin, Taxol and Cisplatin.

16. The composition as claimed in claim 6, wherein cardiovascular drug is selected from the group consisting of Amlodipin, Lisinopril, propranolol and Atenolol.

17. The composition as claimed in claim 6, wherein CNS drugs is selected from the group consisting of Alprazolam and Haloperidol.

18. The composition as claimed in claim 6, wherein anti-inflammatory/anti-arthritic drug is selected from the group consisting of Diclofenac, Piroxicam, Nimesulide and Rofecoxib.

19. The composition as claimed in claim 6, wherein anti-TB/anti-leprosy drug is selected from the group consisting of Rifampicin, Ethionamide, Isoniazid, Cycloserine, Pyrazinamide, Ethambutol and Dapsone.

20. The composition as claimed in claim 6, wherein antihistamine/drugs for respiratory disorders compound is selected from the group consisting of Salbutamol, Theophylline, Bromhexine and Loratidine.

21. The composition as claimed in claim 6, wherein corticosteriod is selected from the group consisting of Prednisolone, dexamethasone and Betamethasone.

22. The composition as claimed in claim 6, wherein immuno-supressant is selected from the group consisting of Cyclosporin A, Tacrolimus and Mycophenolatemofetil.

23. The composition as claimed in claim 6, wherein the anti-ulcer compound is selected from the group consisting of Rantidine, Cimetidine and Omerprazole.

24. The composition as claimed in claim 6, wherein the herbal product/drug is selected from Echinacea, Tinospora cordifolia, Picrorrhiza kurroa, Aegles marmelos, Andrographis paniculata, Emblica ribes, Asparagus racemosus, Terminalia chebula Withania somnifera, Centella asiatica and/or their mixture thereof.

25. The composition as claimed in claim 6, wherein the nutrient is selected from group consisting of sugar, carbohydrates, fats and proteins.

26. The composition as claimed in claim 6, wherein vitamin is selected from the group consisting of Vitamin A, Vitamin E, Vitamin B1, Vitamin B6, Vitamin B12, Vitamin C and Folic acid.

27. The composition as claimed in claim 6, wherein the antioxidant is selected from the group consisting of β-Carotene, Silymarin, Selenium, Lycopene and Ellagiogallotannins.

28. The composition as claimed in claim 6, wherein natural herbal product is selected from the group consisting of Curcumin, Boswellic acids and Rutin.

29. The composition as claimed in claim 6, wherein essential micro nutrients is selected from the group consisting of Methionine, Lysine, Leucine, Valine, Isoleucine, Zinc, Calcium, Glucose, Potassium, Copper and Iron.

30. The composition as claimed in claim 1, wherein, the plant extract of Zingiber officinale or its bioactive fraction or pure isolate is extracted from any plant parts of Zingiber officinale.

31. The composition as claimed in claim 1, wherein said composition is administered through oral, parenteral, nasal, inhalation including nebulisers, rectal, vaginal, transdermal and any others suitable routes.

32. The composition as claimed in claims 1, wherein the bioenhancing effect of the extracts/fractions/pure isolates of Zingiber officinale alone or in combination with piperine is selective in enhancing the bioavailability/bioefficacy of a drug, nutraceutical, and herbal drug/formulation.

33. The composition as claimed in claims 1, wherein the composition comprising components of Zingiber officinale and/or piperine exhibit nearly significant (about 20%) to highly significant (about 200%) bioenhancing/bioavailability activity.

34. The composition as claimed in claim 1, wherein bioavailability/bio-enhancing activity provided by Zingiber officinale alone is up to 75%.

35. The composition as claimed in claim 1, wherein composition containing Zingiber officinale alone provides bioavailability/bio-enhancing activity in the range of 30-75%.

36. The composition as claimed in claim 1, wherein said composition-containing piperine and Zingiber officinale provides further bioavailability/bio-enhancing activity in the range of 10 to 85% beyond Zingiber officinale alone.

37. The composition as claimed in claim 1, wherein the dosage of bioehancer from Zingiber officinale is in the range of 10 to 30-mg/kg/body weight and piperine is in the range of 4 to 12 mg/kg/body weight.

38. The composition as claimed in claim 1, wherein the dosage of bioehancer from Zingiber officinale is in the range of 5 to 15-mg/kg/body weight, preferably 30-mg/kg/body weight, and piperine is in the range of 6 to 10 mg/kg/body weight, preferably 8-mg/kg/body weight.

39. A process for the preparation of an aqueous extract, aq.alcoholic extract and bioactive fraction from the plant Zingiber officinale, said process comprises steps of:
a) extracting crushed plant material with water or aqueous alcoholic solvent at a temperature range of 95-100° C.;
b) cooling and filtering the extract of step (a) to obtain a clear aqeuous extract or aqueous alcoholic extract;
c) evaporating the aq.extract of step (b) under reduced pressure at 60° C. to obtain an concentrated aqueous extract;
d) freeze drying the concentrated aqueous extract of step (c) to obtain a dried aqueous extract;
e) evaporating the solvent from aqueous alcoholic extract of step (b);
f) macerating dried aqueous alocholic extract obtained from step (e) of with chloroform,
g) separating the chloroform soluble fraction from step (f) to obtain fraction 1 and an insoluble fraction;
h) dissolving the insoluble fraction from step (g) in water and adding alcoholic solvent, stirring and filtering the mixture to separate aqueous alcohol soluble portion and a residue;
i) distilling the aqueous alcohol soluble fraction of step (h) under reduced pressure to obtain fraction 2; and
j) dissolving the residue of step (i) in water and freeze drying it to get fraction 3.

40. A process as claimed in claim 39, wherein the alcoholic solvent used is selected from the group consisting of methanol, ethanol, propanol and/or aqueous alcoholic solvent.

41. A process as claimed in claim 39, wherein in step (a) the ratio of plant material to the solvent used is in the range of 1:1 to 1:3, preferably 1:2.5.
